(19) United States
(12) Patent Application Publication
(10) Pub. No.: US 2006/0239995 A1
(43) Pub. Date: Oct. 26, 2006

(54) Silk firing product, antibacterial material using the same and process for producing the silk firing product

(76) Inventors: Hitoshi Hashizume, Chiisagata-gun (JP); Tomio Yawata, Chiisagata-gun (JP); Mutsumi Kimura, Ueda-shi (JP)

Correspondence Address: BIRCH STEWART KOLASCH & BIRCH, PO BOX 747, FALLS CHURCH, VA, 22040-0747, US

(21) Appl. No.: 10/558,307

(22) PCT Filed: Jul. 15, 2004
(86) PCT No.: PCT/JP04/10137

§ 371 Date: Nov. 28, 2005

(30) Foreign Application Priority Data
Jul. 16, 2003 (JP) 2003-275332

(51) Int. Cl. A61K 33/44

(52) U.S. Cl. 424/125; 428/408

(57) ABSTRACT

The present invention provides a novel silk burned product. The silk burned product of the present invention is characterized by the step of burning and carbonizing a silk material at temperature of 1,000° C. or below. By burning at low temperature, nitrogenous components derived from amino acids remain in high proportion. An antibacterial property is exhibited by the remaining of nitrogenous components in high proportion. Accordingly, the silk burned product can be appropriately used as an antibacterial material in a mask, etc..

### FIELD OF TECHNOLOGY

The present invention relates to a silk burned product, which can be appropriately used as an antibacterial material, and a method of producing the product.

### BACKGROUND TECHNOLOGY

In Patent Document 1, a silk burned product, which is produced by burning and carbonizing a silk material, is used as a material for shielding electromagnetic waves.

The silk burned product is formed by the steps of: primary-burning the silk material at low temperature, e.g., 400-450° C.; cooling the silk material; and secondary-burning the silk material at temperature of 1100-1200° C.

Note that, the Patent Document 1 is Japanese Patent Gazette No. 2002-220745.

### DISCLOSURE OF THE INVENTION

The inventors of the present invention studies various uses of silk burned products, and they found that a silk burned product, which was burned at low temperature, i.e., 1000 ° C. or below, had sorbability and an antibacterial property.

The silk burned product of the present invention is characterized by burning and carbonizing a silk material at temperature of 1,000° C. or below.

By burning at low temperature, nitrogenous components derived from amino acids remain in high proportion. The antibacterial property is exhibited by the remaining of nitrogenous components in high proportion.

Accordingly, the silk burned product can be appropriately used as an antibacterial material in a mask, etc..

Preferably, the silk burned product includes 18-35 wt % of nitrogen elements.

The silk burned product may be activation-treated so as to broaden surface area, so that the sorbability and the antibacterial property can be further improved.

The method of the present invention comprises the steps of: primary-burning a silk material with temperature rising rate of 100° C./hour or less until reaching a first temperature; maintaining the first temperature for several hours; secondary-burning the silk material with temperature rising rate of 100° C./hour or less until reaching a second temperature, which is higher than the first temperature and which is 1,000° C. or below; and maintaining the second temperature for several hours, wherein the steps are performed in an inert gas atmosphere.

Preferably, the silk material, which has been primary-burned, is once cooled until reaching the room temperature, then the silk material is secondary-burned.

Preferably, the temperature rising rate in the primary-burning step and the secondary-burning step is 50° C./ hour or less.

Note that, the second temperature for secondary-burning the silk material is 1,000° C. or below; considering flexibility of the silk burned product, the preferable second temperature is 500-900° C., more preferably 600-800° C.

The method may further comprise the step of exposing the silk material, which has been secondary-burned, to high-temperature steam as an activation treatment.

### EFFECTS OF THE INVENTION

In the present invention, the silk burned product is produced by burning and carbonizing the silk material at temperature of 1,000° C. or below so that a glossy black silk burned product, which is not graphitized and which has enough flexibility, can be produced; the silk burned product has a high antibacterial property so that it can be used as an antibacterial material in a mask, etc..

Further, the silk burned product maintains a shape of the silk material; unlike other carbon fibers, the silk burned product is flexible, so it does not stick in a hand.

### BRIEF DESCRIPTION OF THE DRAWINGS

FIG. 1 is A raman spectrum chart of a burned product, which was produced by burning a coarse-grained silk at temperature of 2,000° C.

FIG. 2 is A raman spectrum chart of a burned product, which was produced by burning a coarse-grained silk at temperature of 700° C.

FIG. 3 is A raman spectrum chart of a burned product, which was produced by burning a coarse-grained silk at temperature of 1,000° C.

FIG. 4 is A raman spectrum chart of a burned product, which was produced by burning a coarse-grained silk at temperature of 1,400° C.

FIG. 5 is A FE-SEM photograph of a silk material burned at temperature of 700° C.

FIG. 6 is A FE-SEM photograph of a silk material burned at temperature of 2,000° C.

### PREFERRED EMBODIMENTS OF THE INVENTION

The silk burned product of the present invention is produced by burning a silk material at relatively low temperature, i.e., 1,000° C. or below.

In the following description, silk materials include woven fabrics, knitted works, powders, cloth, strings, etc. made of threads of domesticated or wild silk worms. The silk burned product is produced by burning the silk material or materials.

The silk material should be burned at 1,000° C. or below. A burning atmosphere should be an inert gas atmosphere, e.g., nitrogen gas atmosphere, argon gas atmosphere, or a vacuum atmosphere so as not to burn the silk material to cinders.

The silk material should be burned in stages without rapid burning.

For example, the silk material is primary-burned in the inert gas atmosphere with low temperature rising rate of 1000° C./hour or less, preferably 50 ●/hour or less, until reaching a first temperature (e.g., 500° C.), then the first temperature is maintained for several hours. The silk material is once cooled until reaching the room temperature, then the silk material is secondary-burned in the same atmosphere with low temperature rising rate of 1000/hour or less, preferably 50° C./hour or less, until reaching a second temperature (e.g., 700° C.) and the second temperature is maintained for several hours. Then, the silk material or the silk burned product is cooled until the room temperature and taken out from a furnace. Note that, the silk material may be secondary-burned without cooling after the primary-burning.

Burning conditions are not limited to the above described embodiment, they may be optionally changed on the basis of kinds of silk materials, functions of silk burned products, etc..

By burning the silk material in stages, burning with the low temperature rising rate and burning at 1,000° C. or below, rapid decomposition of a protein high-order structure, in which crystalline forms and noncrystalline forms of a dozen of amino acids are combined, can be avoided, especially variety of functions can be produced by remaining of nitrogenous components in high proportion.

By burning the silk material at low temperature, e.g., 500-1,000° C., the soft (flexible) and glossy black silk burned product can be produced without being graphitized.

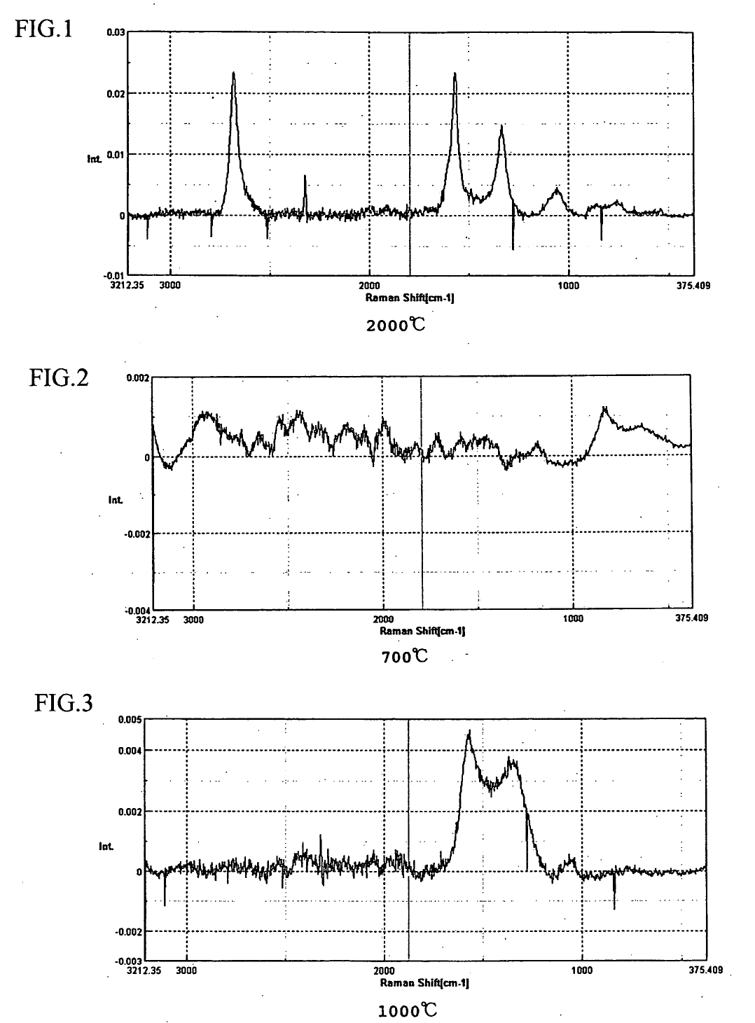

FIG. 1 is a raman spectrum chart of a burned product, which was produced by burning a coarse-grained silk at temperature of 2,000° C. Peaks were observed at 2681 cm⁻¹, 1570 cm⁻¹ and 1335 cm⁻¹, so the coarse-grained silk was graphitized.

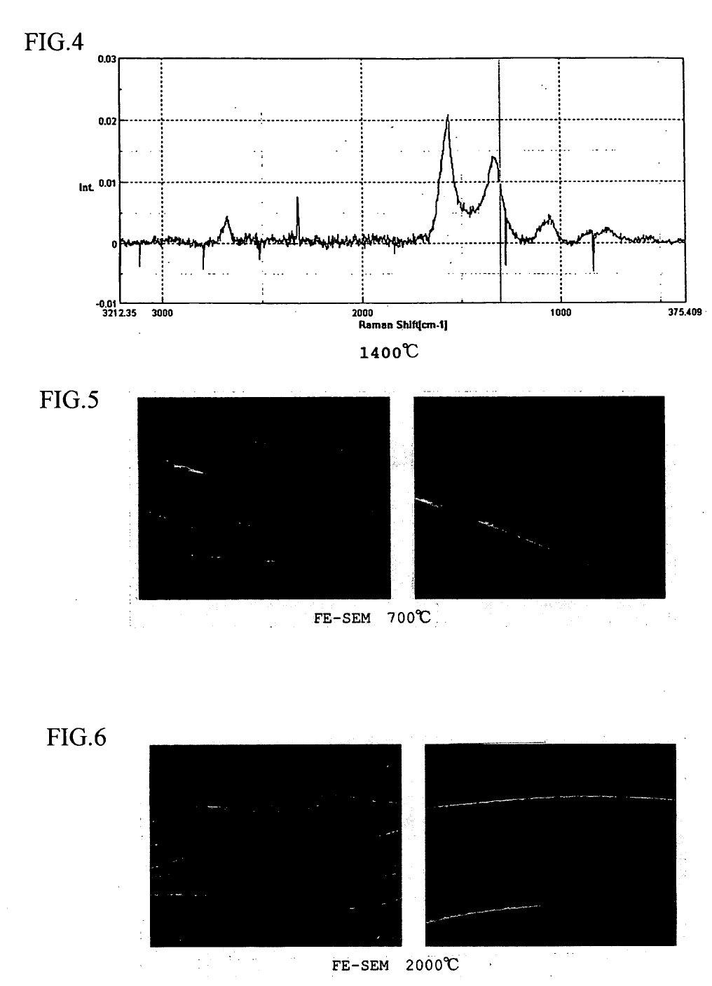

FIGS. 2-4 are raman spectrum charts of burned products, which were produced by respectively burning coarse-grained silks at 700 ●, 1,000 ● and 1,400° C. By burning at 1,400° C., intensities were low but peaks were observed at the same three points. By burning at 1,000° C. or below, no high peaks were observed, so the burned products were not graphitized.

TABLE 1

| | Elements | | | | | | | | | | | | |
|---|---|---|---|---|---|---|---|---|---|---|---|---|---|
| | C | N | O | Na | Mg | Al | Si | P | S | Cl | K | Ca | Fe |
| Wt % | 66.1 | 27.4 | 2.1 | 0.1 | 0.3 | 0.1 | 0.3 | 0.1 | 0.1 | 0.1 | 0.1 | 3.2 | 0.2 |

Table 1 shows results of elemental analysis (semiquantative analysis) of a burned product, which was a knitted work made of silk of domesticated silkworms and which was burned in a nitrogen atmosphere at 700° C., performed by an electron beam micro analyzer.

Measuring conditions were as follows: accelerating voltage was 15 kV; irradiating current was 1 uA; and probe diameter was 100 μm. Note that, values in the table indicate tendency of detected elements but they are not guaranteed values.

According to Table 1, a large amount of nitrogen, i.e., 27.4 wt %, was remained. Further, other elements derived from amino acids were also remained.

TABLE 2

| | Elements | | | | | | | | | | | |
|---|---|---|---|---|---|---|---|---|---|---|---|---|
| | C | N | O | Na | Mg | Al | Si | P | S | Cl | K | Ca |
| Wt % | 74.6 | 15.7 | 5.1 | 0.3 | 0.3 | 0.1 | 0.7 | 0.1 | 0.2 | 0.2 | 0.1 | 2.7 |

Table 2 shows results of elemental analysis (semiquantative analysis) of a burned product, which was a knitted work made of silk of domesticated silkworms and which was burned in a nitrogen atmosphere at 1,400° C., performed by the electron beam micro analyzer.

Measuring conditions were as follows: accelerating voltage was 15 kV; irradiating current was 1 μA; and probe diameter was 100 μm. Note that, values in the table indicate tendency of detected elements but they are not guaranteed values.

According to Table 2, a residual amount of nitrogen was reduced to 15.7 wt %.

TABLE 3

| | Elements | | | | | | | | | | |
|---|---|---|---|---|---|---|---|---|---|---|---|
| | C | N | O | Na | Mg | Al | Si | P | S | K | Ca |
| Wt % | 69.9 | 24.5 | 2.7 | 0.2 | 0.3 | 0.0 | 0.2 | 0.2 | 0.4 | 0.1 | 1.6 |

Table 3 shows results of elemental analysis (semiquantative analysis) of a burned product, which was an unwoven cloth made of silk of domesticated silkworms and which was burned in a nitrogen atmosphere at 700° C., performed by the electron beam micro analyzer.

Measuring conditions were as follows: accelerating voltage was 15 kV; irradiating current was 1 μA; and probe diameter was 100 μm. Note-that, values in the table indicate tendency of detected elements but they are not guaranteed values.

According to Table 3, a large amount of nitrogen, i.e., 24.5 wt %, was remained.

FIG. 5 is a FE-SEM photograph of a silk material burned at temperature of 700° C. A surface of the burned silk material is covered with thin films, which are burned residue derived from amino acids, e.g., nitrogen elements.

On the other hand, FIG. 6 is a FE-SEM photograph of a silk material burned at temperature of 2,000° C., but the surface of the burned silk material is clear and covered with no films.

TABLE 4

| | | | | | Bac- |
|---|---|---|---|---|---|
| | | Number of | | | terio- |
| | | Bacteria | | Sterili- | static |
| | Number of | in Un- | Number of | zation | Ac- |
| | Inoculating | treated | Bacteria | Activity | tivity |
| Bacteria | Bacteria | Cloth | in Sample | Value | Value |
| Staph | 4.3 | 7.1 | 1.3 | 3.0 | 5.8 |
| Bacteria | 2.2E+04 | 1.2E+07 | 2.0E+01 | | |
| Klebsiella | 4.4 | 7.5 | 1.3 | 3.1 | 6.2 |
| Pneumoniae | 2.6E+04 | 3.0E+07 | 2.0E+01 | | |
| MRSA | 4.4 | 7.1 | 1.3 | 3.1 | 5.8 |
| | 2.6E+04 | 1.4E+07 | 2.0E+01 | | |
| Coli | 4.3 | 7.5 | 1.3 | 3.0 | 6.2 |
| Bacteria | 1.8E+04 | 2.9E+07 | 2.0E+01 | | |
| Pseudomonas | 4.2 | 7.0 | 1.3 | 2.9 | 5.7 |
| aeruginosa | 1.7E+04 | 1.1E+07 | 2.0E+01 | | |

Table 4 shows results of antibacterial tests of burned products, which were woven fabrics of domesticated silkworms and which were burned in a nitrogen atmosphere at temperature of 700° C.

The tests were JIS L 1902 quantative tests (unified tests).

Standard cotton cloth was used as the untreated cloth. “Number of Bacteria in Untreated Cloth” means number of bacteria, which have been inoculated and grown in the unburned cloth.

For example, “2.2E+04” in the table is 2.2×10⁴, and the value “4.3” is a logarithm value thereof.

According to Table 4, bacteria considerably grew in the untreated cloth; on the other hand, all bacteria were considerably reduced in samples, i.e., burned cloth, so that we found that the burned product had an antibacterial property.

By burning silk materials in a plurality of stages, burning them at 1,000° C. or below and rising temperature with low temperature rising rate, a large amount of elements derived from amino acids, e.g., nitrogen elements remained in the samples so that the samples could have the antibacterial property.

Since the silk burned product has the antibacterial property, the product can be appropriately used as a material of a mask, etc..

In case of using the silk burned product as an antibacterial filter, a sheet-shaped silk material, which may be woven cloth, knitted fabric, powders or unwoven cloth, is burned to produce a sheet-shaped burned product. By adjusting density of the woven cloth, the knitted fabric, the powders or the unwoven cloth, air permeability of the silk burned product can be adjusted.

An activation treatment may be applied to the silk burned product so as to form irregularities in a surface of the silk burned product and increase surface area thereof, so that the sorbability and the antibacterial property can be improved.

For example, the activation treatment is to expose the silk burned product to high-temperature steam, whose temperature is about 850° C. (1,000° C. or below); as a result of the activation treatment, many micro fine holes (diameters are from 0.1 nm to several dozen nm) are formed in the surface of the silk burned product.

### EXAMPLE 1

A silk material was heated in a nitrogen gas atmosphere until reaching first temperature (450° C.) with low temperature rising rate of 50° C./hour, then the material was burned at the first temperature for five hours as the primary burning. Next, the burned material was once cooled until reaching the room temperature, then the material was reheated in the nitrogen gas atmosphere until reaching second temperature (700° C.) with low temperature rising rate of 50° C./hour, then the material was burned at the second temperature for five hours as the secondary burning. Further, the burned material was cooled until reaching the room temperature, so that the silk burned product shown in FIG. 5 was produced.

The silk burned product was exposed to steam, whose temperature was 850° C., as the activation treatment, so that many micro fine holes were formed in the surface of the silk burned product; the surface area of the silk burned product could be broadened about 1,000 times.

1-10. (canceled)

11. A silk burned product being formed by burning and carbonizing a silk material at temperature of 1,000° C. or below.

12. The silk burned product according to claim 1 1, wherein said silk burned product includes 18-35 wt % of nitrogen elements.

13. The silk burned product according to claim 11, wherein said silk burned product is activation-treated so as to form many micro fine holes in a surface thereof.

14. The silk burned product according to claim 12, wherein said silk burned product is activation-treated so as to form many micro fine holes in a surface thereof.

15. An antibacterial material being made of a silk burned product, which is formed by burning and carbonizing a silk material at temperature of 1,000° C. or below.

16. The antibacterial material according to claim 15, wherein said silk burned product includes 18-35 wt % of nitrogen elements.

17. The antibacterial material according to claim 16, wherein said silk burned product is activation-treated so as to form many micro fine holes in a surface thereof.

18. The antibacterial material according to claim 17, wherein said silk burned product is activation-treated so as to form many micro fine holes in a surface thereof.

19. A method of producing a silk burned product comprising the steps of: primary-burning a silk material with temperature rising rate of 100° C./hour or less until reaching a first temperature; maintaining the first temperature for several hours; secondary-burning the silk material with temperature rising rate of 100° C./hour or less until reaching a second temperature, which is higher than the first temperature and which is 1,000° C. or below; and maintaining the second temperature for several hours, wherein said steps are performed in an inert gas atmosphere.

20. The method according to claim 19, wherein the silk material, which has been primary-burned, is once cooled until reaching the room temperature, then the silk material is secondary-burned.

21. The method according to claim 20, wherein the temperature rising rate in the primary-burning step and the secondary-burning step is 50° C./hour or less.

22. The method according to claim 19, further comprising the step of exposing the silk material, which has been secondary-burned, to high-temperature steam as an activation treatment.

23. The method according to claim 20, further comprising the step of exposing the silk material, which has been secondary-burned, to high-temperature steam as an activation treatment.

24. The method according to claim 1, further comprising the step of exposing the silk material, which has been secondary-burned, to high-temperature steam as an activation treatment.
